(19) United States
(12) Patent Application Publication
(10) Pub. No.: US 2003/0094173 A1
(43) Pub. Date: May 22, 2003

(54) Aerosolization device with improved endpiece connection

(75) Inventors: John D. Burr, Redwood City, CA (US); Jeff R. Wood, Mountain View, CA (US); Adrian E. Smith, Belmont, CA (US); John A. Howard, Palo Alto, CA (US)

Correspondence Address: NEKTAR THERAPEUTICS, 150 INDUSTRIAL ROAD, SAN CARLOS, CA, 94070, US

(73) Assignee: Inhale Therapeutic Systems, Inc., San Carlos, CA

(21) Appl. No.: 10/295,783

(22) Filed: Nov. 14, 2002

Related U.S. Application Data
(60) Provisional application No. 60/336,541, filed on Nov. 14, 2001.

(51) Int. Cl. A61M 11/00; B65D 83/06

(52) U.S. Cl. 128/200.23; 222/394

(57) ABSTRACT

An aerosolization apparatus comprises a body having an inlet, an endpiece having an outlet, the endpiece being connectable to the body to define a chamber, wherein the chamber is sized to receive a capsule containing a pharmaceutical formulation in a manner which allows the capsule to move within the chamber. A connection mechanism is provided to provide selective connection of the endpiece to the body, and a locking member prevents undesired disconnection of the endpiece from the body. When a user inhales, air enters into the chamber through the inlet so that the pharmaceutical formulation is aerosolized within the chamber and the aerosolized pharmaceutical formulation is delivered to the user through the outlet. In another version, the endpiece and the body are hinged together.

### RELATED APPLICATIONS

The present application claims the benefit of U.S. Provisional Application No. 60/336,541 filed on Nov. 14, 2001.

### BACKGROUND

The need for effective therapeutic treatment of patients has resulted in the development of a variety of pharmaceutical formulation delivery techniques. One traditional technique involves the oral delivery of a pharmaceutical formulation in the form of a pill, capsule, elixir, or the like. However, oral delivery can in some cases be undesirable. For example, many pharmaceutical formulations may be degraded in the digestive tract before they can be effectively absorbed by the body. Inhaleable drug delivery, where an aerosolized pharmaceutical formulation is orally or nasally inhaled by a patient to deliver the formulation to the patient's respiratory tract, has proven to be a particularly effective and/or desirable alternative. For example, in one inhalation technique, a pharmaceutical formulation is delivered deep within a patient's lungs where it may be absorbed into the blood stream. Many types of inhalation devices exist including devices that aerosolize a dry powder, devices comprising a pharmaceutical formulation stored in or with an inhaleable propellant, devices which use a compressed gas to aerosolize a liquid pharmaceutical formulation, and similar devices.

In one dry powder aerosolization technique, a capsule containing an inhaleable dry powder is loaded into a chamber in an aerosolization device. Within the chamber, the dry powder is at least partially emptied and dispersed to aerosolize the dry powder so that it may be inhaled by a patient. However, in conventional devices, the manner of accessing the chamber may often lead to device inconsistencies and/or failures. Also, the dry powder in the cavity can cause the access mechanism to become less effective at efficiently opening and closing.

Therefore, it is desirable to improve the manner of accessing an aerosolization device chamber. It is further desirable to access the chamber in a manner that reduces device inconsistencies and/or failures. It is still further desirable to access the cavity so that debris in the cavity will have reduced adverse affects on the functioning of the device.

### SUMMARY

The present invention satisfies these needs. In one aspect of the invention an aerosolization apparatus comprises a body and an endpiece, the body and endpiece being connectable to one another by a connection mechanism. The aerosolization apparatus further comprises locking member to prevent inadvertent disconnection of the parts.

In another aspect of the invention, an aerosolization apparatus comprises a body having an inlet; an endpiece having an outlet, the endpiece being connectable to the body to define a chamber, wherein the chamber is sized to receive a capsule containing a pharmaceutical formulation in a manner which allows the capsule to move within the chamber; a connection mechanism to provide selective connection of the endpiece to the body; a locking member to prevent undesired disconnection of the endpiece from the body; and a puncturing mechanism capable of providing an opening in the capsule; whereby when a user inhales, air enters into the chamber through the inlet so that the pharmaceutical formulation is aerosolized within the chamber and the aerosolized pharmaceutical formulation is delivered to the user through the outlet.

In another aspect of the invention, an aerosolization apparatus comprises a body having an inlet; an endpiece having an outlet, the endpiece being connectable to the body to define a chamber, wherein the chamber is sized to receive a capsule containing a pharmaceutical formulation in a manner which allows the capsule to move within the chamber; a connection mechanism to provide selective connection of the endpiece to the body; a blocking member positionable in a first position where the flow of air through the inlet is at least partially blocked and a second position where the flow of air through the inlet is less blocked; and a puncturing mechanism capable of providing an opening in the capsule; whereby when a user inhales, air enters into the chamber through the inlet so that the pharmaceutical formulation is aerosolized within the chamber and the aerosolized pharmaceutical formulation is delivered to the user through the outlet.

In another aspect of the invention, an aerosolization apparatus comprises a body having an inlet; an endpiece having an outlet, the endpiece being connectable to the body to define a chamber, wherein the chamber is sized to receive a capsule containing a pharmaceutical formulation in a manner which allows the capsule to move within the chamber; a connection mechanism to provide selective connection of the endpiece to the body; a flexible member to tether the endpiece to the body; and a puncturing mechanism capable of providing an opening in the capsule; whereby when a user inhales, air enters into the chamber through the inlet so that the pharmaceutical formulation is aerosolized within the chamber and the aerosolized pharmaceutical formulation is delivered to the user through the outlet.

In another aspect of the invention, an aerosolization apparatus comprises a body having an inlet; an endpiece having an outlet, the endpiece being connectable to the body to define a chamber having a longitudinal axis, wherein the chamber is sized to receive a capsule containing a pharmaceutical formulation in a manner which allows the capsule to move within the chamber; a connection mechanism to provide selective connection of the endpiece to the body, wherein a rotational force is needed to connect or disconnect the endpiece and the body, the rotational force being applied about an axis which is not parallel to the longitudinal axis of the chamber; and a puncturing mechanism capable of providing an opening in the capsule; whereby when a user inhales, air enters into the chamber through the inlet so that the pharmaceutical formulation is aerosolized within the chamber and the aerosolized pharmaceutical formulation is delivered to the user through the outlet.

In another aspect of the invention, a method of aerosolizing a pharmaceutical formulation comprises inserting a capsule containing a pharmaceutical formulation into a chamber in a body; connecting an endpiece to the body; locking the endpiece to the body; before, during, or after inserting the capsule into the chamber, providing an opening in the capsule; and inhaling through an opening in the endpiece to cause air to flow into the chamber through an inlet in the body thereby aerosolizing the pharmaceutical formulation.

In another aspect of the invention, a method of aerosolizing a pharmaceutical formulation comprises inserting a capsule containing a pharmaceutical formulation into a chamber in a body; at least partially blocking an inlet in the body; connecting an endpiece to the body; unblocking the inlet in the body; before, during, or after inserting the capsule into the chamber, providing an opening in the capsule; and inhaling through an opening in the endpiece to cause air to flow into the chamber through the inlet in the body thereby aerosolizing the pharmaceutical formulation.

In another aspect of the invention, a method of aerosolizing a pharmaceutical formulation comprises inserting a capsule containing a pharmaceutical formulation into a chamber in a body; rotating an endpiece relative to the body to connect the endpiece to the body, the rotation being about an axis which is not parallel to the longitudinal axis of the chamber; before, during, or after inserting the capsule into the chamber, providing an opening in the capsule; and inhaling through an opening in the endpiece to cause air to flow into the chamber through an inlet in the body thereby aerosolizing the pharmaceutical formulation.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a schematic sectional side view of a version of an aerosolization device of the invention with an endpiece and body connected;

FIG. 2 is a schematic sectional side view of the version of an aerosolization device of FIG. 1 with the endpiece and body disconnected;

FIG. 3 is a schematic sectional side view of a version of an aerosolization device in use;

FIGS. 4A and 4B are schematic side views of another version of an aerosolization device in disconnected and connected configurations, respectively;

FIG. 5 is a schematic sectional view of a version of an aerosolization device;

FIG. 6 is a schematic sectional view of a version of an aerosolization device;

FIGS. 7A and 7B are schematic side views of another version of an aerosolization device in disconnected and connected configurations, respectively; and

FIGS. 8A and 8B are schematic side views of another version of an aerosolization device in disconnected and connected configurations, respectively.

### DESCRIPTION

The present invention relates to delivering an aerosolized pharmaceutical formulation to a patient. Although the process is illustrated in the context of aerosolizing a dry powder pharmaceutical formulation, the present invention can be used in other processes and should not be limited to the examples provided herein.

An aerosolization device 100 of the present invention is shown schematically in FIG. 1. The aerosolization device 100 includes a body 105 and an endpiece 110 that may be attached to the body 105 to form a chamber 115 within the interior of the body 105 and the endpiece 110. The endpiece 110 includes an end 120 defining an outlet 125. The end 120 may be sized and shaped to be received in a user's mouth. Alternatively, the end 120 may be sized and shaped to be received in a nostril of a user or may sized and shaped to be received by a mask, a spacer chamber, a respirator circuit, or the like. The body includes one or more inlets 130 in communication with the chamber 115. Together the inlets 130, the chamber 115, and the outlet 125 define an airway through the aerosolization device 100. Accordingly, when a user contacts the endpiece 110 and inhales or otherwise creates a vacuum at the outlet 125, a pharmaceutical formulation within the chamber 115 may be delivered to the user through the outlet 125. The pharmaceutical formulation may be contained within a container that is positionable within the chamber 115. In one version, the pharmaceutical formulation may be contained within a capsule that is positionable within the chamber 115, the chamber 115 being sized to receive the capsule in a manner which allows the capsule to move within the chamber 115. In this version, the endpiece 110 includes a perforated member 135 having one or more openings 140 therein. The perforated member 135 sufficiently blocks the chamber 115 to retain a capsule in the chamber 115, while the openings 140 allow air and/or other material to pass to the outlet 125. A connection mechanism 150 may be provided to allow the endpiece 110 to be attached to the body 105.

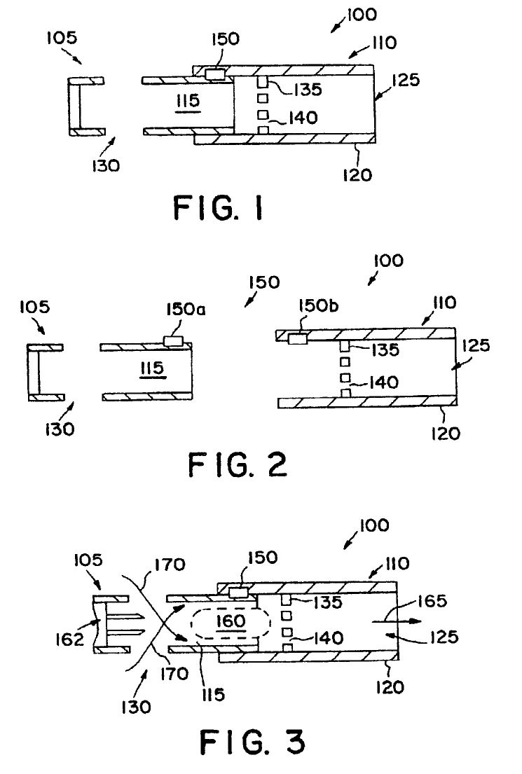

In one version, as shown in FIG. 2, the connection mechanism 150 may allow the body 105 and the endpiece 110 to be disconnected to allow for access to the chamber 115. In this version, the endpiece 110 may be disconnected from the body 105 to allow a pharmaceutical formulation to be inserted into the chamber, for example by allowing a capsule to be inserted into the chamber 115. In this version, the connection mechanism includes a body connection member 150a that cooperates with an endpiece connection member 150b to selectively connect and disconnect the endpiece 110 to the body 105.

After a capsule 160 has been inserted into the chamber 115, the endpiece 110 may again be attached to the body 105 to secure the capsule 160 within the chamber 115, as shown in FIG. 3. The capsule 160 is opened, for example by puncturing the capsule 160 prior to insertion or within the chamber 115, such as by longitudinally advancing a sliding puncture mechanism 162.

When opened, the pharmaceutical formulation in the capsule is allowed to exit the capsule 160. In one version, the pharmaceutical formulation is in a dry powder form and the flow of air through the airway causes the pharmaceutical formulation to be aerosolized. For example, as shown in FIG. 3, a user may contact the endpiece 110 with his or her mouth and inhale, thereby drawing air through the outlet 125, as shown by arrow 165. This inhalation causes air to be taken in through the inlets 130, as shown by arrows 170. The air taken in causes the capsule 160 to agitate within the chamber 115. The agitation causes the dry powder pharmaceutical formulation to leave the capsule 160 and become aerosolized in the airway. The aerosolized pharmaceutical formulation passes through the perforated member 135 and is delivered to the user where it may be inhaled to a position in the user's respiratory tract. In one particular embodiment, a plurality of inlets 130 may be designed to cause the inlet air 170 to swirl within the chamber, for example, by being at least partially tangentially oriented as described in U.S. Pat. No. 4,995,385 and U.S. Pat. No. 4,069,819, both of which are incorporated herein by reference in their entireties. In such an arrangement, the chamber 115 comprises a longitudinal axis that lies generally in the inhalation direction 165, and the capsule 160 is insertable lengthwise into the chamber 115 so that the capsule's longitudinal axis may be parallel to the longitudinal axis of the chamber 115. The swirling air flow then causes the capsule to rotate within the chamber 115 in a manner where the longitudinal axis of the capsule is remains at an angle less than 80 degrees, and preferably less than 45 degrees from the longitudinal axis of the chamber. In one version, this rotation is caused by the width of the chamber being less than the length of the capsule.

Often, a user will grasp the body 105 during use while inhaling through the endpiece 110. It has been discovered that doing so may create a disconnection force in the inhalation direction 165 between the body 105 and the endpiece 110. Accordingly, the connection mechanism 150 may be designed to prevent undesired disconnection of the endpiece 110 from the body 105 during use.

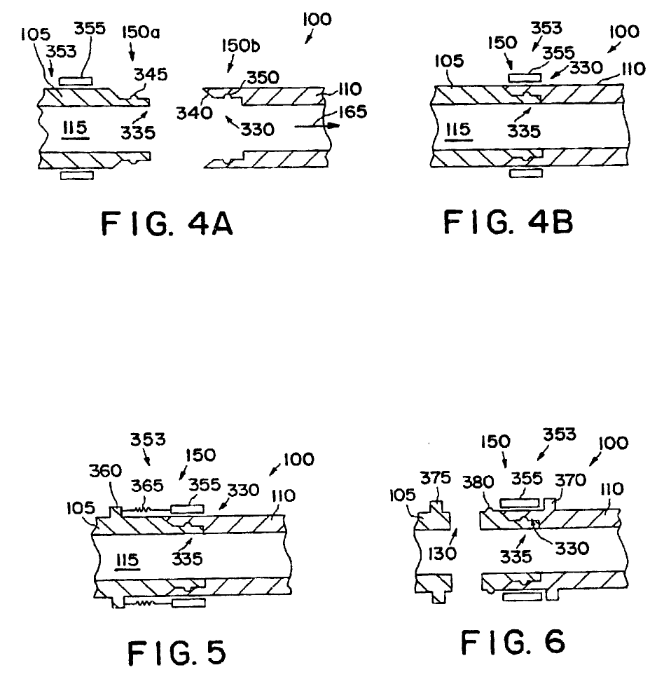

In one version, the connection mechanism 150 includes a locking member which prevents undesired disconnection of the endpiece 110 from the body 105. Accordingly, a force in the inhalation direction 165 is insufficient to disconnect the endpiece 110 from the body 105 when the locking member is in a locking position. Examples of connection mechanisms of this type are schematically shown in FIGS. 4-6.

In the version of FIGS. 4A and 4B, the endpiece 110 comprises a female portion 330 that snap fits onto a male portion 335 on the body 105. In the version shown, a projection 345 is received within a recess 350 on the female portion 330 to provide the snap fit. Optionally, the female portion 330 may include a sloped surface 340 to facilitate insertion of the female portion over the projection 345. Thus, as the female portion 330 is inserted over the male portion 335, the female portion expands a sufficient amount to pass over the projection 345 until the recess 350 is in alignment with the projection 345. A locking member 353 may be provided to prevent inadvertent disconnection of the endpiece 110 from the body 105. For example, the locking member 353 may comprise a slidable sleeve 355 that may be positioned around the female portion 330, as shown in FIG. 4B. In the locked position of FIG. 4B, the sleeve 355 prevents the female portion 330 from expanding and thereby locks the projection 345 within the recess 350.

In another version, the locking member 353 is positioned in the locking position during use. For example, in the version shown in FIG. 5, the locking member 353 may be biased into the locked position. For example, a compressed spring 365 may bias the sleeve 355 away from a collar 360 on the body 105. With this version, the user manually displaces the sleeve 355 against the bias of the spring 365 in order to connect the parts or disconnect the parts. By biasing the sleeve 355 into the locked position, inadvertent disconnection as a result of the user forgetting to move the sleeve into a locked position is avoided. In another version, such as the version of FIG. 6, the sleeve 355 may be slidable between collars 370, 375 and may be positioned in either a locked or an unlocked position, the positions being separated by a projection 380 on the outer surface of the body 105. The projection 380 is of sufficient size to allow the user to slide the sleeve 355 thereover but to provide enough resistance to indicate to the user that the sleeve is positioned on the projection 380 instead of in one of the positions on either side of the projection 380. In the particular version shown, the sleeve 355 may be positioned over the inlets 130 when in the unlocked position. Thus, when unlocked, the sleeve serves as a blocking member to at least partially block the inlets 130 to prevent air to flow through the inlets 130, substantially preventing unlocked use of the aerosolization device 100.

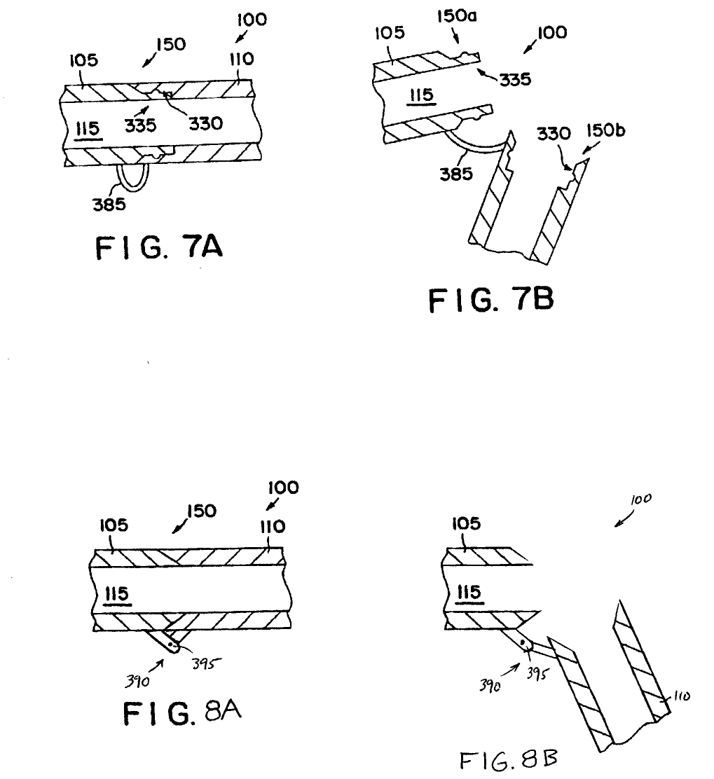

Another version of an aerosolization device 100 is shown in FIGS. 7A and 7B and FIGS. 8A and 8B. In these versions, the body 105 and the endpiece 110 remain attached to one another, even when disconnected. This attachment further prevents the endpiece 105 from being inhaled by a user. In the version of FIGS. 7A and 7B, the body 105 and the endpiece 110 are connected by a flexible connector 385. The flexible connector 385 operate as a hinge about which the body 105 and the endpiece 110 may pivot relative to one another. Alternatively, the flexible connector may merely tether the endpiece 110 to the body 105 to prevent complete separation of the parts. In another version, the flexible connector 385 may be biased into the configuration shown in FIG. 7A. In the biased version, the flexible connector 385 helps prevent undesired separation of the endpiece 110 from the body 105. In the version of FIGS. 8A and 8B, the body 105 and the endpiece 110 are hinged together be a hinge 390 comprising a pivot pin 395 about which the body 105 and the endpiece 110 relatively rotate. As can be seen, in the version shown the relative rotation is about an axis substantially perpendicular to the longitudinal axis of the chamber in order to prevent inadvertent disconnection of the parts. The hinge 390 may include a bias member to bias the endpiece 110 toward the body 105. In the version shown, the connection does not include a snap fit connection. In another version, the snap fit connection is included with the hinge 390.

In a preferred version, the invention provides a system and method for aerosolizing a pharmaceutical formulation and delivering the pharmaceutical formulation to the lungs of the user. The pharmaceutical formulation may comprise powdered medicaments, liquid solutions or suspensions, and the like, and may include an active agent.

The active agent described herein includes an agent, drug, compound, composition of matter or mixture thereof which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable active agents may be selected from, for example, hypnotics and sedatives, psychic energizers, tranquilizers, respiratory drugs, anticonvulsants, muscle relaxants, antiparkinson agents (dopamine antagonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of active agents suitable for use in this invention include but are not limited to one or more of calcitonin, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIIa inhibitor, alpha-1 antitrypsin, phosphodiesterase (PDE) compounds, VLA-4 inhibitors, bisphosponates, respiratory syncytial virus antibody, cystic fibrosis transmembrane regulator (CFTR) gene, deoxyreibonuclease (Dnase), bactericidal/permeability increasing protein (BPI), anti-CMV antibody, 13-cis retinoic acid, macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate, polymixins such as polymixin B, capreomycin, bacitracin, penems; penicillins including penicillinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments and analogs thereof.

Active agents for use in the invention further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense. Further, an active agent may comprise live attenuated or killed viruses suitable for use as vaccines. Other useful drugs include those listed within the Physician's Desk Reference (most recent edition).

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 99% by weight active agent, typically from about 2% to about 95% by weight active agent, and more typically from about 5% to 85% by weight active agent, and will also depend upon the relative amounts of additives contained in the composition. The compositions of the invention are particularly useful for active agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one active agent may be incorporated into the formulations described herein and that the use of the term “agent” in no way excludes the use of two or more such agents.

The pharmaceutical formulation may comprise a pharmaceutically acceptable excipient or carrier which may be taken into the lungs with no significant adverse toxicological effects to the subject, and particularly to the lungs of the subject. In addition to the active agent, a pharmaceutical formulation may optionally include one or more pharmaceutical excipients which are suitable for pulmonary administration. These excipients, if present, are generally present in the composition in amounts ranging from about 0.01% to about 95% percent by weight, preferably from about 0.5 to about 80%, and more preferably from about 1 to about 60% by weight.

Preferably, such excipients will, in part, serve to further improve the features of the active agent composition, for example by providing more efficient and reproducible delivery of the active agent, improving the handling characteristics of powders, such as flowability and consistency, and/or facilitating manufacturing and filling of unit dosage forms. In particular, excipient materials can often function to further improve the physical and chemical stability of the active agent, minimize the residual moisture content and hinder moisture uptake, and to enhance particle size, degree of aggregation, particle surface properties, such as rugosity, ease of inhalation, and the targeting of particles to the lung. One or more excipients may also be provided to serve as bulking agents when it is desired to reduce the concentration of active agent in the formulation.

Pharmaceutical excipients and additives useful in the present pharmaceutical formulation include but are not limited to amino acids, peptides, proteins, non-biological polymers, biological polymers, carbohydrates, such as sugars, derivatized sugars such as alditols, aldonic acids, esterified sugars, and sugar polymers, which may be present singly or in combination. Suitable excipients are those provided in WO 96/32096, which is incorporated herein by reference in its entirety. The excipient may have a glass transition temperatures (Tg) above about 35° C., preferably above about 40° C., more preferably above 45° C., most preferably above about 55° C.

Exemplary protein excipients include albumins such as human serum albumin (HSA), recombinant human albumin (rHA), gelatin, casein, hemoglobin, and the like. Suitable amino acids (outside of the dileucyl-peptides of the invention), which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and polypeptides that function as dispersing agents. Amino acids falling into this category include hydrophobic amino acids such as leucine, valine, isoleucine, tryptophan, alanine, methionine, phenylalanine, tyrosine, histidine, and proline. Dispersibility-enhancing peptide excipients include dimers, trimers, tetramers, and pentamers comprising one or more hydrophobic amino acid components such as those described above.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol and the like.

The pharmaceutical formulation may also include a buffer or a pH adjusting agent, typically a salt prepared from an organic acid or base. Representative buffers include organic acid salts of citric acid, ascorbic acid, gluconic acid, carbonic acid, tartaric acid, succinic acid, acetic acid, or phthalic acid, Tris, tromethamine hydrochloride, or phosphate buffers.

The pharmaceutical formulation may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-β-cyclodextrin and sulfobutylether-β-cyclodextrin), polyethylene glycols, and pectin.

The pharmaceutical formulation may further include flavoring agents, taste-masking agents, inorganic salts (for example sodium chloride), antimicrobial agents (for example benzalkonium chloride), sweeteners, antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), both of which are incorporated herein by reference in their entireties.

“Mass median diameter” or “MMD” is a measure of mean particle size, since the powders of the invention are generally polydisperse (i.e., consist of a range of particle sizes). MMD values as reported herein are determined by centrifugal sedimentation, although any number of commonly employed techniques can be used for measuring mean particle size. “Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. As used herein, MMAD refers to the midpoint or median of the aerodynamic particle size distribution of an aerosolized powder determined by cascade impaction.

In one version, the powdered formulation for use in the present invention includes a dry powder having a particle size selected to permit penetration into the alveoli of the lungs, that is, preferably 10 μm mass median diameter (MMD), preferably less than 7.5 μm, and most preferably less than 5 μm, and usually being in the range of 0.1 μm to 5 μm in diameter. The delivered dose efficiency (DDE) of these powders may be greater than 30%, more preferably greater than 40%, more preferably greater than 50% and most preferably greater than 60% and the aerosol particle size distribution is about 1.0-5.0 μm mass median aerodynamic diameter (MMAD), usually 1.5-4.5 μm MMAD and preferably 1.5-4.0 μm MMAD. These dry powders have a moisture content below about 10% by weight, usually below about 5% by weight, and preferably below about 3% by weight. Such powders are described in WO 95/24183, WO 96/32149, WO 99/16419, and WO 99/16422, all of which are all incorporated herein by reference in their entireties.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the cooperating components may be reversed or provided in additional or fewer number. Also, the various features of the versions herein can be combined in various ways to provide additional versions of the present invention. Furthermore, certain terminology has been used for the purposes of descriptive clarity, and not to limit the present invention. Therefore, the appended claims should not be limited to the description of the preferred versions contained herein and should include all such alterations, permutations, and equivalents as fall within the true spirit and scope of the present invention.

What is claimed is:

1. An aerosolization apparatus comprising:
a body having an inlet;
an endpiece having an outlet, the endpiece being connectable to the body to define a chamber, wherein the chamber is sized to receive a capsule containing a pharmaceutical formulation in a manner which allows the capsule to move within the chamber;
a connection mechanism to provide selective connection of the endpiece to the body;
a locking member to prevent undesired disconnection of the endpiece from the body; and
a puncturing mechanism capable of providing an opening in the capsule;
whereby when a user inhales, air enters into the chamber through the inlet so that the pharmaceutical formulation is aerosolized within the chamber and the aerosolized pharmaceutical formulation is delivered to the user through the outlet.

2. An apparatus according to claim 1 wherein the connection mechanism comprises a snap fit connection between the endpiece and the body.

3. An apparatus according to claim 2 wherein the snap fit connection comprises a projection on one of the body and the endpiece, the projection being receivable within a recess in the other of the body and the endpiece.

4. An apparatus according to claim 3 wherein the locking member is positionable to prevent the removal of the projection from within the recess.

5. An apparatus according to claim 1 wherein the locking member is positionable to prevent disconnection of the connection mechanism.

6. An apparatus according to claim 1 wherein the locking member comprises a sleeve that is positionable in a locking position and an unlocking position.

7. An apparatus according to claim 6 wherein the locking member is biased toward the locking position.

8. An apparatus according to claim 6 wherein the sleeve is slidable between the locking position and the unlocking position.

9. An apparatus according to claim 8 further comprising a projection between the locking position and the unlocking position.

10. An apparatus according to claim 6 wherein the locking member at least partially blocks an inlet when in the unlocking position.

11. An aerosolization apparatus according to claim 1 wherein the inlet is shaped to provide a swirling air flow in the chamber.

12. An apparatus according to claim 1 wherein the chamber is elongated and has a longitudinal axis, and wherein the longitudinal axis of the chamber and the longitudinal axis of the capsule form an angle of less than about 45 degrees during use.

13. An apparatus according to claim 1 wherein the chamber is elongated and wherein the capsule is received lengthwise within the elongated chamber.

14. An aerosolization apparatus according to claim 1 wherein the width of the chamber is less than the length of the capsule.

15. An aerosolization apparatus comprising:
a body having an inlet;
an endpiece having an outlet, the endpiece being connectable to the body to define a chamber, wherein the chamber is sized to receive a capsule containing a pharmaceutical formulation in a manner which allows the capsule to move within the chamber;
a connection mechanism to provide selective connection of the endpiece to the body;
a blocking member positionable in a first position where the flow of air through the inlet is at least partially blocked and a second position where the flow of air through the inlet is less blocked; and
a puncturing mechanism capable of providing an opening in the capsule;
whereby when a user inhales, air enters into the chamber through the inlet so that the pharmaceutical formulation is aerosolized within the chamber and the aerosolized pharmaceutical formulation is delivered to the user through the outlet.

16. An apparatus according to claim 15 wherein the blocking member comprises a sleeve.

17. An apparatus according to claim 15 wherein the blocking member prevents disconnection of the endpiece from the body when in the second position.

18. An aerosolization apparatus comprising:
a body having an inlet;
an endpiece having an outlet, the endpiece being connectable to the body to define a chamber, wherein the chamber is sized to receive a capsule containing a pharmaceutical formulation in a manner which allows the capsule to move within the chamber;
a connection mechanism to provide selective connection of the endpiece to the body;
a flexible member to tether the endpiece to the body; and
a puncturing mechanism capable of providing an opening in the capsule;
whereby when a user inhales, air enters into the chamber through the inlet so that the pharmaceutical formulation is aerosolized within the chamber and the aerosolized pharmaceutical formulation is delivered to the user through the outlet.

19. An apparatus according to claim 18 wherein the flexible member biases the endpiece toward the body.

20. An aerosolization apparatus comprising:
a body having an inlet;
an endpiece having an outlet, the endpiece being connectable to the body to define a chamber having a longitudinal axis, wherein the chamber is sized to receive a capsule containing a pharmaceutical formulation in a manner which allows the capsule to move within the chamber;
a connection mechanism to provide selective connection of the endpiece to the body, wherein a rotational force is needed to connect or disconnect the endpiece and the body, the rotational force being applied about an axis which is not parallel to the longitudinal axis of the chamber; and
a puncturing mechanism capable of providing an opening in the capsule;
whereby when a user inhales, air enters into the chamber through the inlet so that the pharmaceutical formulation is aerosolized within the chamber and the aerosolized pharmaceutical formulation is delivered to the user through the outlet.

21. An apparatus according to claim 20 wherein a rotational force about an axis substantially perpendicular to the longitudinal axis of the chamber is needed to connect or disconnect the endpiece and the body.

22. An apparatus according to claim 20 wherein the connection mechanism comprises a hinge which rotatably connects the endpiece and the body.

23. An apparatus according to claim 22 wherein the hinge comprises a flexible member that tethers the endpiece to the body.

24. An apparatus according to claim 22 wherein the hinge comprises a pivot pin about which the endpiece and the body relatively rotate.

25. A method of aerosolizing a pharmaceutical formulation, the method comprising:
inserting a capsule containing a pharmaceutical formulation into a chamber in a body;
connecting an endpiece to the body;
locking the endpiece to the body;
before, during, or after inserting the capsule into the chamber, providing an opening in the capsule; and
inhaling through an opening in the endpiece to cause air to flow into the chamber through an inlet in the body thereby aerosolizing the pharmaceutical formulation.

26. A method according to claim 25 wherein the endpiece is connected to the body by a snap fit connection.

27. A method according to claim 26 wherein the step of locking the endpiece to the body comprises preventing disconnection of the snap fit connection.

28. A method of aerosolizing a pharmaceutical formulation, the method comprising:
inserting a capsule containing a pharmaceutical formulation into a chamber in a body;
at least partially blocking an inlet in the body;
connecting an endpiece to the body;
unblocking the inlet in the body;
before, during, or after inserting the capsule into the chamber, providing an opening in the capsule; and
inhaling through an opening in the endpiece to cause air to flow into the chamber through the inlet in the body thereby aerosolizing the pharmaceutical formulation.

29. A method according to claim 28 wherein the step of unblocking the inlet simultaneously prevents disconnection of the endpiece and the body.

30. A method of aerosolizing a pharmaceutical formulation, the method comprising:
inserting a capsule containing a pharmaceutical formulation into a chamber in a body;
rotating an endpiece relative to the body to connect the endpiece to the body, the rotation being about an axis which is not parallel to the longitudinal axis of the chamber;
before, during, or after inserting the capsule into the chamber, providing an opening in the capsule; and
inhaling through an opening in the endpiece to cause air to flow into the chamber through an inlet in the body thereby aerosolizing the pharmaceutical formulation.

31. A method according to claim 30 wherein the rotation is about an axis substantially perpendicular to the longitudinal axis of the chamber.
